(19) United States
(12) Patent Application Publication
(10) Pub. No.: US 2026/0189604 A1
(43) Pub. Date: Jul. 2, 2026

(54) Hardware-Anchored Diagnostic Genesis and Multimodal Sensor-Level Cryptographic Sealing System for Zero-Trust AI Acquisition

(71) Applicant: Geprge William Bickerstaff, III, Greenwich, CT (US)

(72) Inventor: Geprge William Bickerstaff, III, Greenwich, CT (US)

(21) Appl. No.: 19/546,361

(22) Filed: Feb. 22, 2026

(51) Int. Cl. H04L 9/40; G06F 21/86; G16H 40/67; H04L 9/32

(52) U.S. Cl. CPC H04L 63/1466; G06F 21/86; G16H 40/67; H04L 9/3278

(57) ABSTRACT

A hardware-anchored cryptographic provenance system for securing raw medical data precisely at the analog-to-digital conversion edge. The system integrates a physical unclonable function directly into medical diagnostic equipment, such as MRI scanners or ECG monitors. At the exact millisecond of biological data capture, an analog-to-digital provenance anchor generates a cryptographic seal that binds the raw data to the device's unforgeable hardware identity, creating a Diagnostic Genesis Package. This inbound data sealing prevents the injection of maliciously generated synthetic deepfakes or altered imagery into downstream artificial intelligence networks. A zero-trust ingestion gateway at the receiving server mathematically verifies the sensor seal, physically dropping any data lacking legitimate hardware provenance. The system supports multimodal cross-sensor synchronization and tamper-responsive zeroization, ensuring that downstream AI shadow execution and global federated training models ingest only pristine, mathematically verified patient data.

### FIELD

The present invention relates to hardware-secured data acquisition in medical diagnostic equipment. More particularly, the invention provides a silicon-anchored cryptographic provenance system embedded directly into analog-to-digital converters of medical sensors to mathematically seal raw biological data at the exact millisecond of capture, physically neutralizing adversarial data tampering at the Tissue-to-Digital Trust Boundary before transmission to downstream artificial intelligence processing environments.

### BACKGROUND

As hospitals deploy advanced continuous-learning artificial intelligence, the absolute purity of the incoming patient data becomes the foundational vulnerability of the entire ecosystem. Existing medical imaging and telemetry devices transmit raw data across hospital networks where it is highly susceptible to man-in-the-middle attacks, data poisoning, and the injection of maliciously generated synthetic imagery (deepfakes). There is an unmet need for a hardware architecture that shifts the root of trust completely out of the server environment and directly into the physical medical scanner, mathematically sealing the analog-to-digital conversion process to create an unforgeable genesis record for downstream AI execution and federated aggregation.

### PRACTICAL APPLICATION & NON-OBVIOUSNESS

The claimed invention integrates cryptographic sealing into a specific, hardware-secured analog-to-digital architecture that materially alters the physical sensor's processor state to execute inbound data sealing. By utilizing a physical unclonable function embedded within the scanner, this system physically rejects synthetic deepfakes and man-in-the-middle attacks at sub-millisecond speeds, as demonstrated in Example 1. This significantly mitigates the catastrophic risks of adversarial data injection into downstream diagnostic pipelines, providing a quantifiable technological improvement over generic software network security that satisfies 35 U.S.C. § 101. Furthermore, this hardware integration addresses long-felt, unmet cybersecurity needs for verifiable data provenance at the point of clinical capture, providing strong secondary indicia of non-obviousness that explicitly bolsters the Graham factors under 35 U.S.C. § 103.

### DEFINITIONS

Analog-to-Digital Provenance Anchor: The localized secure processor physically embedded directly onto the medical scanner's internal analog-to-digital converter. It manages all cryptographic operations at the exact physical point where biological signals become digital code. It completely bypasses the scanner's standard operating system to prevent software-level interference during data creation.

Diagnostic Genesis Package: An unforgeable digital payload containing the raw medical data, a physical hardware signature, and a precise temporal timestamp. It serves as the absolute baseline of truth for any downstream artificial intelligence evaluating the patient. It physically cannot be generated by any system other than an authorized medical sensor.

Multimodal Synchronization Lock: A specialized cryptographic token that securely aligns data from two different physical sensors taken simultaneously. It mathematically proves that a patient's blood pressure drop occurred at the exact same millisecond as their recorded heart arrhythmia. It provides downstream artificial intelligence models with perfectly synchronized context for complex multimodal diagnoses.

Physical Unclonable Function (PUF) Emitter: The silicon fingerprint generator built directly into the medical scanner's hardware. It translates microscopic silicon manufacturing variations into a unique cryptographic identity for the specific hospital machine. It physically prevents malicious actors from spoofing expensive medical equipment on the hospital network.

Sensor-Level Hash Chain: A permanent, sequential digital ledger maintained directly inside the physical memory of the medical device itself. It securely records the exact time and mathematical signature of every single scan the machine performs. It provides an indisputable forensic audit trail proving the scanner's historical operational integrity.

Spatial-Temporal Context Seal: A cryptographic mechanism that locks the exact geographic location and atomic-clock timestamp to the diagnostic data. It proves that the scan was taken in an authorized hospital room at a specific moment in time. It prevents attackers from submitting old, stolen medical records as fresh clinical data.

Synthetic Deepfake Rejector: A hardware logic gate at the receiving artificial intelligence server that instantly drops any medical imagery lacking a valid Genesis Package. It mathematically recognizes that flawless, AI-generated cancer nodules will always lack the required physical hardware signature of a true scanner. It physically blocks these dangerous digital fabrications before they can trick downstream diagnostic algorithms.

Tamper-Responsive Zeroization Circuit: A physical defense mechanism surrounding the secure hardware components inside the medical scanner. It constantly monitors for unauthorized chassis breaches, extreme thermal fluctuations, or anomalous electrical currents. It irreversibly destroys all cryptographic keys within one microsecond if physical tampering is detected.

Tissue-to-Digital Trust Boundary: The highly vulnerable physical threshold where organic patient biology is converted into a digital machine format. The system focuses entirely on securing this specific microscopic gap to prevent analog signal interception. It represents the absolute first line of defense in the medical artificial intelligence lifecycle.

Zero-Trust Ingestion Gateway: The fiercely protected receiving terminal located on downstream artificial intelligence servers or federated aggregation nodes. It fundamentally assumes all incoming network traffic is malicious until mathematically proven otherwise. It demands an unbroken Diagnostic Genesis Package before passing data to local shadow execution environments or global training models.

### BRIEF DESCRIPTION OF THE DRAWINGS

Referring now to the drawings submitted separately in compliance with 37 CFR 1.84, the following figures illustrate the preferred embodiments of the invention.

### DETAILED DESCRIPTION OF THE DRAWINGS

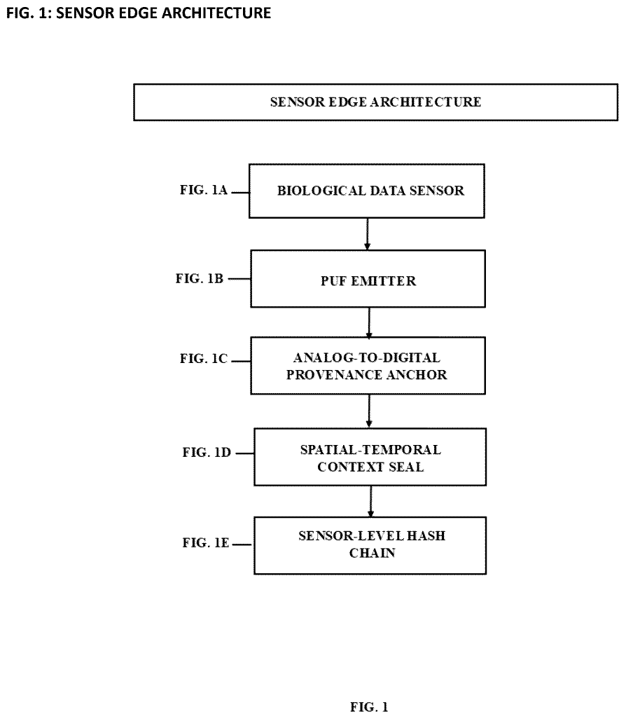

### FIG. 1: Sensor Edge Architecture

Referring now to FIG. 1A, the Biological Data Sensor physically interacts with the patient to capture raw physiological signals like heart rhythms or tissue densities. It utilizes highly sensitive analog components to measure these critical biological metrics in real time. It serves as the absolute origin point for the entire diagnostic artificial intelligence lifecycle.

Referring now to FIG. 1B, the PUF Emitter translates microscopic silicon manufacturing variations into an unforgeable cryptographic identity for the medical scanner. It physically guarantees that the data originated from a specific, authorized hospital machine rather than a hacker's software simulation. It prevents malicious actors from remotely spoofing expensive medical equipment on the hospital network.

Referring now to FIG. 1C, the Analog-to-Digital Provenance Anchor houses a secure processor directly on the medical scanner's internal conversion circuitry. It acts as the localized root of trust to manage cryptographic sealing at the exact point of analog translation. It completely bypasses the scanner's standard software to prevent privilege escalation attacks from capturing unencrypted data.

Referring now to FIG. 1D, the Spatial-Temporal Context Seal interfaces with a secure atomic time feed and localized proximity beacons. It locks the exact geographic coordinates and precise millisecond of the scan directly into the data file. It mathematically prevents an adversary from submitting a stolen, ten-year-old MRI scan as a new patient record.

Referring now to FIG. 1E, the Sensor-Level Hash Chain maintains a permanent digital ledger directly inside the medical device's physical memory. It sequentially records the unique mathematical signature of every single patient scan the machine ever performs. It provides an indisputable forensic audit trail for hospital administrators and federal regulatory inspectors.

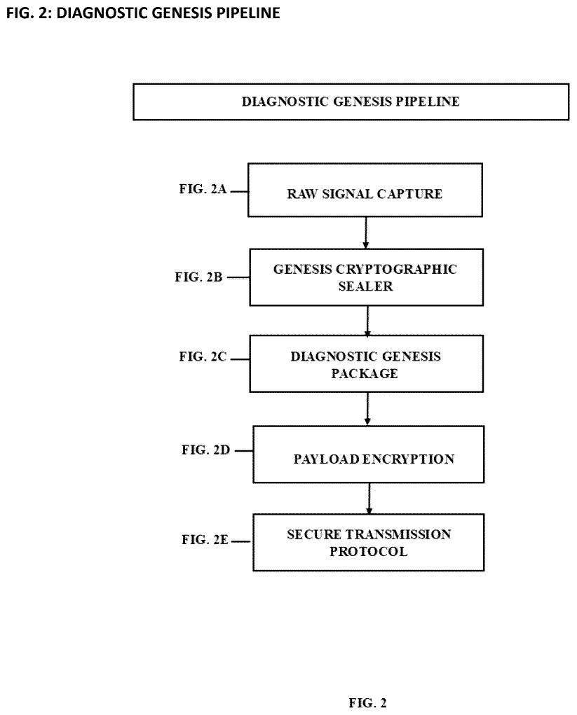

### FIG. 2: Diagnostic Genesis Pipeline

Referring now to FIG. 2A, the Raw Signal Capture module activates the precise moment the medical imaging or scanning procedure begins. It immediately funnels the unfiltered biological data into the physically isolated hardware enclave. It ensures no external network connections can access the data during the highly vulnerable Tissue-to-Digital Trust Boundary phase.

Referring now to FIG. 2B, the Genesis Cryptographic Sealer permanently binds the scanner's unique hardware identity to the newly captured medical file. It utilizes advanced cryptographic mathematics to lock the data file before it ever touches a network cable. This physical seal mathematically guarantees the diagnostic image has not been artificially generated by deepfake technology.

Referring now to FIG. 2C, the Diagnostic Genesis Package compiler wraps the raw data, the hardware signature, and the timestamp into a single payload. It formats this payload to be universally readable by authorized downstream artificial intelligence pipelines. It creates the ultimate, unalterable baseline of truth required for safe medical diagnostics.

Referring now to FIG. 2D, the Payload Encryption engine wraps the Genesis Package in an additional layer of military-grade network encryption. It secures the patient's sensitive health information for its upcoming transit across the local hospital intranet. It strictly utilizes session keys derived directly from the physical unclonable function to ensure maximum transit security.

Referring now to FIG. 2E, the Secure Transmission Protocol actively establishes a protected digital tunnel to downstream artificial intelligence servers. It securely routes the encrypted, sealed data packets away from standard hospital web traffic. It guarantees the safe delivery of the pristine medical data to subsequent shadow execution or global training systems.

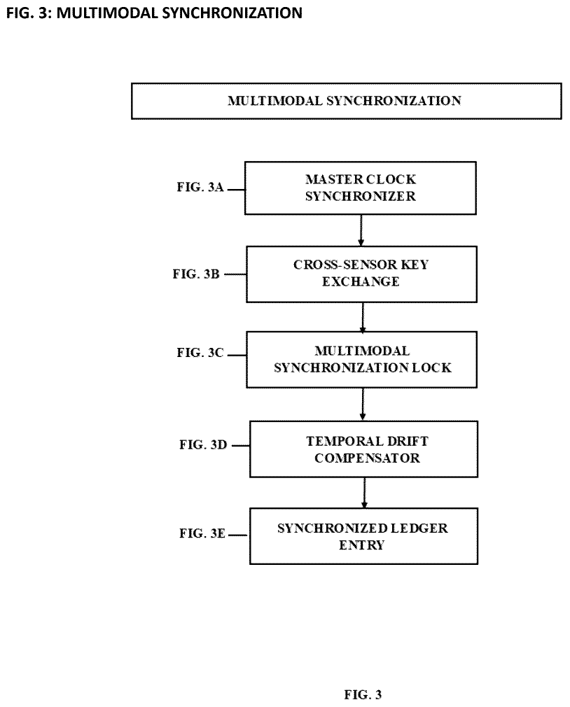

### FIG. 3: Multimodal Synchronization

Referring now to FIG. 3A, the Master Clock Synchronizer ensures all disparate hospital edge devices perfectly agree on the exact current time. It utilizes a secure, hardware-isolated time feed to completely neutralize malicious network time-shifting attacks. It guarantees that the temporal data attached to multiple simultaneous medical scans is perfectly aligned.

Referring now to FIG. 3B, the Cross-Sensor Key Exchange allows two entirely separate medical machines to securely handshake with each other. It enables an electrocardiogram monitor and a localized ultrasound machine to mathematically link their data streams in real time. It encrypts this highly sensitive machine-to-machine communication to prevent local network snooping.

Referring now to FIG. 3C, the Multimodal Synchronization Lock seamlessly binds the outputs from multiple sensors into one unified file. It links the disparate data streams together using a master cryptographic seal generated by a designated primary device. It provides downstream artificial intelligence with a perfectly synchronized, multi-angle snapshot of the patient's acute health.

Referring now to FIG. 3D, the Temporal Drift Compensator corrects microscopic analog timing errors that naturally occur between different physical machines. It mathematically realigns the data streams so downstream AI models do not misinterpret the exact sequence of biological events. It ensures absolute diagnostic precision when evaluating rapid physiological cascading failures.

Referring now to FIG. 3E, the Synchronized Ledger Entry permanently records the successful multimodal data capture in the hospital's central database. It mathematically links the unique hardware identities of every medical sensor involved in the complex procedure. It creates a flawless historical record of the diagnostic event for future liability or research referencing.

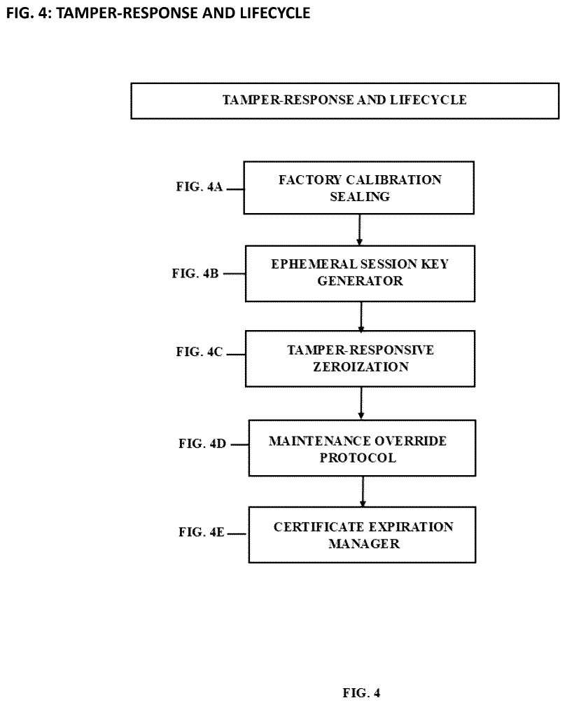

FIG. 4: Tamper-Response and Lifecycle

Referring now to FIG. 4A, the Factory Calibration Sealing process occurs before the medical scanner ever leaves the original manufacturing facility. It permanently burns the foundational cryptographic keys directly into the device's physical silicon architecture. It establishes the absolute baseline of physical trust that the hospital will rely upon for the machine's entire lifespan.

Referring now to FIG. 4B, the Ephemeral Session Key Generator creates temporary, single-use passwords for every individual patient scan. It mathematically guarantees that if one scan is theoretically decrypted, past or future scans remain completely secure. It constantly rotates these complex cryptographic keys to frustrate persistent network cyberattacks.

Referring now to FIG. 4C, the Tamper-Responsive Zeroization circuit acts as the ultimate physical defense mechanism for the medical device hardware. It actively monitors the scanner's internal casing for unauthorized physical access or anomalous thermal changes. It irreversibly destroys all cryptographic keys within one microsecond if a physical tampering attempt is definitively detected.

Referring now to FIG. 4D, the Maintenance Override Protocol provides authorized repair technicians with secure, temporary access to the medical scanner internals. It requires a mathematically verified digital token from the original manufacturer to temporarily pause the zeroization security functions. It meticulously logs every single action the technician takes in the permanent hardware ledger.

Referring now to FIG. 4E, the Certificate Expiration Manager automatically revokes the scanner's data sealing capabilities if mandatory safety calibrations expire. It physically forces the hospital to properly maintain their medical equipment according to strict federal regulations. It prevents degraded analog sensors from feeding dangerously inaccurate biological data to diagnostic AI models.

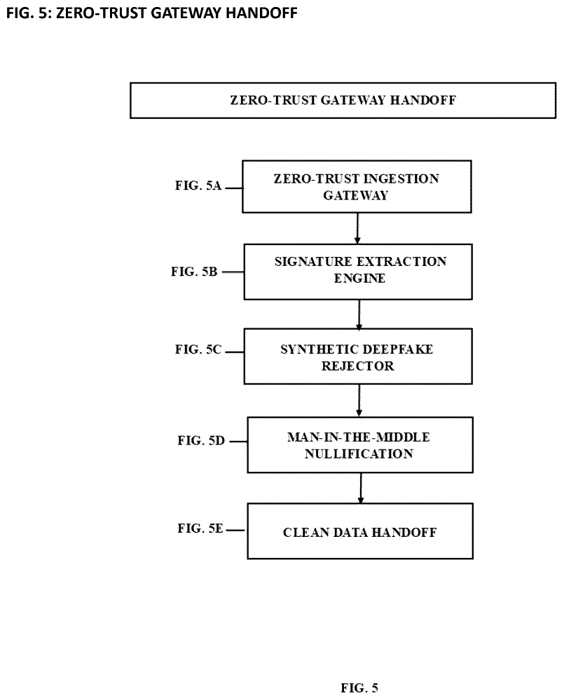

FIG. 5: Zero-Trust Gateway Handoff

Referring now to FIG. 5A, the Zero-Trust Ingestion Gateway acts as the fiercely protected entry point for any downstream artificial intelligence processing server. It fundamentally assumes all incoming network traffic is malicious until mathematically proven otherwise. It physically blocks any medical data from entering an AI pipeline unless the hardware signature perfectly matches.

Referring now to FIG. 5B, the Signature Extraction Engine mathematically deconstructs the incoming file to examine the Genesis Package seal. It confirms that the cryptographic signature perfectly matches the registered physical identity of an authorized, calibrated medical scanner. It instantly rejects the file if even a single digital bit of the medical image has been altered in transit.

Referring now to FIG. 5C, the Synthetic Deepfake Rejector specifically targets and eliminates maliciously generated artificial intelligence imagery. It recognizes that flawless, AI-generated tumors will always lack the required physical hardware seal of a real scanner. It physically drops these dangerous digital fabrications before they can corrupt local shadow testing or global federated training models.

Referring now to FIG. 5D, the Man-in-the-Middle Nullification circuit detects if a data packet was intercepted and delayed during local network transit. It instantly flags files that arrive with mathematically irregular timestamps or broken sequential hash chains. It completely destroys the corrupted data payload to prevent network-based tampering from affecting live patient care.

Referring now to FIG. 5E, the Clean Data Handoff finally releases the verified, pristine Genesis Package to the authorized downstream artificial intelligence environments. It provides the subsequent aggregation engines and local inference models with absolute mathematical certainty regarding data purity. It is the critical final step that enables legally defensible, zero-trust medical AI diagnostics.

### Examples Of Enablement

Example 1: Neutralizing Synthetic Deepfakes Before Local AI Ingestion. A malicious actor breaches a hospital's internal network and attempts to inject a synthetic, AI-generated deepfake MRI showing a false hemorrhage into the hospital's diagnostic queue to trigger an unnecessary surgery. However, the hospital's MRI machines are equipped with the Analog-to-Digital Provenance Anchor. Because the deepfake was generated on a hacker's laptop and not the physical MRI machine, it entirely lacks the physical PUF Emitter's signature. When the deepfake reaches the central AI server's Zero-Trust Ingestion Gateway, the Synthetic Deepfake Rejector instantly identifies the missing Diagnostic Genesis Package. The gateway physically drops the malicious payload in under one millisecond, ensuring the downstream AI models process only pristine, authentic patient imagery.

### Example 2: Synchronized Multimodal Capture for Downstream

Federated Learning. A patient in a critical care unit is connected to a continuous blood pressure monitor and a multi-lead ECG. Utilizing the Cross-Sensor Key Exchange, both physical devices establish a synchronized secure session. As both sensors capture data simultaneously, their respective Analog-to-Digital Provenance Anchors mathematically bind the two analog data streams together using a Multimodal Synchronization Lock. This creates a unified Diagnostic Genesis Package that is securely transmitted to a downstream federated learning node. The downstream node receives mathematically guaranteed proof that the blood pressure drop and the electrical heart anomaly occurred at the exact same millisecond, providing perfectly correlated training data to improve the global AI model without any risk of temporal desynchronization.

What is claimed:

1. A hardware-secured diagnostic acquisition system for establishing zero-trust data provenance at the analog-to-digital boundary, comprising: a biological data sensor configured to capture raw physiological signals; an analog-to-digital converter; an analog-to-digital provenance anchor comprising a localized secure processor physically embedded directly upon said converter; and a physical unclonable function emitter configured to generate a mathematically unique hardware identity, wherein the provenance anchor is hardwired to append a cryptographic seal derived directly from the physical unclonable function to the raw physiological signals at the exact millisecond of digital conversion to generate an unforgeable diagnostic genesis package within a hardware-isolated secure enclave prior to any network transmission.

2. A method for neutralizing adversarial data injection and synthetic deepfakes prior to artificial intelligence processing, comprising the steps of: receiving an incoming diagnostic data file at a zero-trust ingestion gateway located on a downstream artificial intelligence server; utilizing a signature extraction engine to mathematically extract a hardware-embedded cryptographic seal from the data file;
verifying the cryptographic seal against a registry of authorized physical medical sensor hardware identities; and executing a synthetic deepfake rejector comprising a physical hardware logic gate to irreversibly drop the data file before artificial intelligence ingestion if the cryptographic seal is invalid, synthetically generated, or absent.

3. A multimodal cross-sensor synchronization system for generating
unified diagnostic genesis packages, comprising: a first medical diagnostic acquisition device comprising a first analog-to-digital provenance anchor; a second medical diagnostic acquisition device comprising a second analog-to-digital provenance anchor; a master clock synchronizer; and a cross-sensor key exchange protocol, wherein the first and second provenance anchors establish a synchronized cryptographic session to mathematically bind simultaneous physiological data streams into a unified diagnostic genesis package sealed by a shared multimodal synchronization lock, guaranteeing absolute temporal alignment for downstream artificial intelligence analysis.

4. The system of claim 1, wherein the analog-to-digital provenance anchor operates entirely independently of the medical diagnostic acquisition system's primary software operating system, thereby completely preventing software-level privilege escalation attacks from capturing unsealed physiological signals.

5. The system of claim 1, further comprising a sensor-level hash chain configured to sequentially and permanently record the unique mathematical signature of every scan performed directly within the physical, tamper-responsive memory of the acquisition system.

6. The system of claim 1, further comprising a tamper-responsive zeroization circuit physically hardwired to irreversibly destroy all cryptographic session keys within one microsecond upon detection of unauthorized physical casing breach, anomalous electrical current, or extreme temperature deviation.

7. The method of claim 2, further comprising the step of executing a man-in-the-middle nullification protocol that actively monitors network transit timestamps and instantly destroys any data payload exhibiting mathematical temporal irregularities indicative of network interception.

8. The method of claim 2, wherein the zero-trust ingestion gateway processes and verifies the diagnostic genesis package in under one millisecond to prevent transmission latency in highly acute clinical care environments.

9. The method of claim 2, wherein the physical dropping of data lacking the valid diagnostic genesis package directly resolves long-felt cybersecurity needs by completely neutralizing the threat of AI-generated medical image tampering before the data enters downstream shadow execution or global training environments.

10. The system of claim 3, wherein the temporal drift compensator utilizes hardware-isolated atomic time feeds to mathematically realign microscopic analog timing errors between the first and second medical diagnostic acquisition devices.

11. The system of claim 3, wherein the unified diagnostic genesis package guarantees that downstream continuous learning artificial intelligence models cannot misinterpret the precise sequential order of rapid biological events across multiple physiological systems.

12. The system of claim 1, further comprising a spatial-temporal context seal configured to cryptographically lock the exact geographic coordinates of the physical scanner into the diagnostic genesis package to prevent the unauthorized submission of historically captured data.
